(19) United States
(12) Patent Application Publication
(10) Pub. No.: US 2004/0106797 A1
(43) Pub. Date: Jun. 3, 2004

(54) Novel xanthines having adenosine A1-receptor antagonist properties

(76) Inventor: Pascal Druzgala, Santa Rosa, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/624,713

(22) Filed: Jul. 21, 2003

Related U.S. Application Data
(60) Provisional application No. 60/397,099, filed on Jul. 19, 2002.

(51) Int. Cl. C07D 473/02

(52) U.S. Cl. 544/267

(57) ABSTRACT

The subject invention provides 1,3-dipropylxanthines that have an ester function at the 8-position also have potent and specific A₁AdoR antagonist properties.

### CROSS-REFERENCE TO RELATED APPLICATION

This application claims the benefit of U.S. Provisional Application No. 60/397,099, filed Jul. 19, 2002.

### BACKGROUND OF INVENTION

Renal function is a very important prognostic indicator in patients with congestive heart failure. Recently, it has been shown that adenosine may mediate much kidney activity. In addition to vasoconstrictive and vasodilatory effects, adenosine is intrinsic to the tubuloglomerular feedback which occurs when an acute increase in sodium levels in the proximal tubule feeds back to decrease glomerular filtration.

Adenosine works via both adenosine A1 and A2 receptors. A1-receptor antagonists decrease afferent arteriolar pressure, and increase urine flow and sodium excretion.

Certain 8-substituted 1,3-dipropylxanthines, such as KF-15372, KW-3902, KFM-19, and CVT-124, induce diuresis by inhibiting the renal A₁ adenosine receptor (A₁AdoR). These compounds represent a valuable class of diuretic agents in that A1AdoR blockade reduces proximal reabsorption and uncouples it from glomerular filtration. Therefore they do not decrease glomerular filtration, unlike other diuretics such as furosemide that elicit their pharmacological action through a different mechanism. Another important consequence of A₁AdoR blockade is the conservation of potassium ions and the elimination of sodium. These properties make this class of compounds extremely valuable in people with congestive heart failure (CHF).

### BRIEF SUMMARY

The subject invention provides novel 1,3-dipropylxanthines that have an ester function at the 8-position and that have potent and specific A₁AdoR antagonist properties. Because of the presence of the ester function, and unlike other previously described 8-substituted xanthines that do not have an ester function at the 8-position, these compounds are primarily metabolized by non-oxidative enzymes and their primary metabolites are inactive. Because esterases are ubiquitous and do not depend on cytochrome P450 (CYP450), these compounds are not subject to drug-drug interactions and therefore are intrinsically safe for human use.

The compounds of the subject invention are particularly advantageous because of their water solubility and favorable metabolic profile. Specifically, these compounds are readily metabolized by hydrolytic enzymes. Thus, these compounds which have a highly predictable pharmokinetic profile are particularly advantageous because they reduce systemic exposure to the active drug.

The present invention also provides methods of treatment which involve administering an effective amount of a compound of the present invention to a person in need of such treatment.

### BRIEF DESCRIPTION OF DRAWINGS

FIG. 1 shows a method of synthesizing compounds of the subject invention (i) Ac₂O/AcONa (ii) NaNO₂/HCl (iii) Na₂S₂O₄/NH₄OH (iv) R-COCl/TEA.

FIGS. 2A-2S show examples of compounds having A₁AdoR antagonist activity.

### DETAILED DISCLOSURE

The subject invention provides novel A₁, AdoR antagonists. In a preferred embodiment, the antagonists of the subject invention can be deactivated to a primary inactive metabolite by hydrolytic enzymes. Compounds of the present invention also have advantageous water solubility characteristics. These compounds can be advantageously used to treat individuals suffering from congestive heart failure (CHF). The compounds of the subject invention are particularly advantageous because they have more predictable pharmacokinetics and reduced systemic exposure of the drugs.

As used herein, the term “individual(s)” refers to a mammal to which is administered a compound or composition of the present invention. The mammal may be, for example a mouse, rat, pig, horse, rabbit, goat, pig, cow, cat, dog, or human. In a preferred embodiment, the individual is a human.

In accordance with the subject invention, it has been determined that 1,3-dipropylxanthines that have an ester function at the 8-position also have potent and specific A₁AdoR antagonist properties. Because of the presence of the ester function, and unlike other previously described 8-substitutes xanthines that do not have an ester function at the 8-position, these compounds are primarily metabolized by non-oxidative enzymes and their primary metabolites are inactive. Because esterases are ubiquitous and do not depend on cytochrome P450 (CYP450), these compounds are not subject to drug-drug interactions and therefore are intrinsically safe for human use.

Adverse drug-drug interactions (DDI), elevation of liver function test (LFT) values, and QT prolongation leading to torsades de pointes (TDP) are three major reasons why drug candidates fail to obtain FDA approval. All these causes are, to some extent, metabolism-based. A drug that has two metabolic pathways, one oxidative and one non-oxidative, built into its structure is highly desirable in the pharmaceutical industry. An alternate, non-oxidative metabolic pathway provides the treated subject with an alternative drug detoxification pathway (an escape route) when one of the oxidative metabolic pathways becomes saturated or non-functional. While a dual metabolic pathway is necessary in order to provide an escape metabolic route, other features are needed to obtain drugs that are safe regarding DDI, TDP, and LFT elevations.

In addition to having two metabolic pathways, the drug should have a rapid metabolic clearance (short metabolic half-life) so that blood levels of unbound drug do not rise to dangerous levels in cases of DDI at the protein level. Also, if the metabolic half-life of the drug is too long, then the CYP450 system again becomes the main elimination pathway, thus defeating the original purpose of the design. In order to avoid high peak concentrations and rapidly declining blood levels when administered, such a drug should also be administered using a delivery system that produces constant and controllable blood levels over time.

The compounds of this invention have one or more of the following characteristics or properties:

1. Compounds of the invention are metabolized both by CYP450 and by a non-oxidative metabolic enzyme or system of enzymes;

2. Compounds of the invention have a short (up to four (4) hours) non-oxidative metabolic half-life;

3. Oral bioavailability of the compounds is consistent with oral administration using standard pharmaceutical oral formulations; however, the compounds, and compositions thereof, can also be administered using any delivery system that produces constant and controllable blood levels over time;

4. Compounds according to the invention contain a hydrolysable bond that can be cleaved non-oxidatively by hydrolytic enzymes;

5. Compounds of the invention can be made using standard techniques of small-scale and large-scale chemical synthesis;

6. The primary metabolites of compounds of this invention results from the non-oxidative metabolism of the compounds;

7. The primary metabolites, regardless of the solubility properties of the parent drug, is, or are, soluble in water at physiological pH and have, as compared to the parent compound, a significantly reduced pharmacological activity;

8. The primary metabolites, regardless of the electrophysiological properties of the parent drug, has, or have, negligible inhibitory activity at the IK_{R} (HERG) channel at normal therapeutic concentration of the parent drug in plasma (e.g., the concentration of the metabolite must be at least five times higher than the normal therapeutic concentration of the parent compound before activity at the IK_{R} channel is observed);

9. Compounds of the invention, as well as the metabolites thereof, do not cause metabolic DDI when co-administered with other drugs;

10. Compounds of the invention, as well as metabolites thereof, do not elevate LFT values when administered alone.

In some embodiments, the subject invention provides compounds have any two of the above-identified characteristics or properties. Other embodiments provide for compounds having at least any three of the above-identified properties or characteristics. In another embodiment, the compounds, and compositions thereof, have any combination of at least four of the above-identified characteristics or properties. Another embodiment provides compounds have any combination of five to 10 of the above-identified characteristics or properties. In a preferred embodiment the compounds of the invention have all ten characteristics or properties.

In various embodiments, the primary metabolites of the inventive compounds, regardless of the electrophysiological properties of the parent drug, has, or have, negligible inhibitory activity at the IK_{R} (HERG) channel at normal therapeutic concentrations of the drug in plasma. In other words, the concentration of the metabolite must be at least five times higher than the normal therapeutic concentration of the parent compound before activity at the IK_{R} channel is observed. Preferably, the concentration of the metabolite must be at least ten times higher than the normal therapeutic concentration of the parent compound before activity at the IK_{R} channel is observed.

Compounds according to the invention are, primarily, metabolized by endogenous hydrolytic enzymes via hydrolysable bonds engineered into their structures. The primary metabolites resulting from this metabolic pathway are water soluble and do not have, or show a reduced incidence of, DDI when administered with other medications (drugs). Non-limiting examples of hydrolysable bonds that can be incorporated into compounds according to the invention include amide, ester, carbonate, phosphate, sulfate, urea, urethane, glycoside, or other bonds that can be cleaved by hydrolases.

Additional modifications of the compounds disclosed herein can readily be made by those skilled in the art. Thus, analogs and salts of the exemplified compounds are within the scope of the subject invention. With a knowledge of the compounds of the subject invention skilled chemists can use known procedures to synthesize these compounds from available substrates. As used in this application, the term “analogs” refers to compounds which are substantially the same as another compound but which may have been modified by, for example, adding additional side groups. The term “analogs” as used in this application also may refer to compounds which are substantially the same as another compound but which have atomic or molecular substitutions at certain locations in the compound.

The subject invention further pertains to enantiomerically isolated compounds, and compositions comprising the compounds, for A₁AdoR antagonism. The isolated enantiomeric forms of the compounds of the invention are substantially free from one another (i.e., in enantiomeric excess). In other words, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. In one embodiment of the invention, the isolated enantiomeric compounds are at least about in 80% enantiomeric excess. In a preferred embodiment, the compounds are in at least about 90% enantiomeric excess. In a more preferred embodiment, the compounds are in at least about 95% enantiomeric excess. In an even more preferred embodiment, the compounds are in at least about 97.5% enantiomeric excess. In a most preferred embodiment, the compounds are in at least 99% enantiomeric excess.

A further aspect of the subject invention pertains to the breakdown products which are produced when the therapeutic compounds of the subject invention are acted upon by hydrolytic enzymes, such as esterases. The presence of these breakdown products in urine or serum can be used to monitor the rate of clearance of the therapeutic compound from a patient.

The compounds of this invention have therapeutic properties similar to those of the unmodified parent compounds. Accordingly, dosage rates and routes of administration of the disclosed compounds are similar to those already used in the art and known to the skilled artisan (see, for example, Physicians' Desk Reference. 54^{th} Ed., Medical Economics Company, Montvale, N.J., 2000).

The compounds of the subject invention can be formulated according to known methods for preparing pharmaceutically useful compositions. Formulations are described in detail in a number of sources, which are well known and readily available to those skilled in the art. For example, Remington's Pharmaceutical Science by E. W. Martin describes formulation, which can be used in connection with the subject invention. In general, the compositions of the subject invention are formulated such that an effective amount of the bioactive compound(s) are present in the composition.

In accordance with the subject invention, pharmaceutical compositions are provided which comprise, as an active ingredient, an effective amount of one or more of the compounds and one or more non-toxic, pharmaceutically acceptable carriers or diluents.

Compounds of the present invention may be formulated as solutions or suspensions, in the form of tablets, capsules (each including timed release and sustained release formulations), pills, oils, powders, granules, elixers, tinctures, suspensions, syrups, emulsions, microemulsions, or with excipients. Likewise, they may also be administered by any conventional route, for example in intravenous (both bolus and infusion), intraperitoneal, intraocularly, subcutaneous, intramuscular form, enterally, preferably orally (e.g., in the form of tablets or capsules), or in a nasal, buccal, transdermal, or a suppository form, using well known formulations to those of ordinary skill in the pharmaceutical arts.

In addition, the compounds of the present invention can also be administered in the form of liposomes or the like. Disintegrators include, without limitation, delivery systems such as small unilamellar vesicles, large unilamellar vesicles and multilamellar vesicles. Liposomes can be formed from a variety of phospholipids, such as cholesterol, stearylamine or phosphatidylcholines.

For oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, for example, sodium oleate, sodium stearate, magnesium stearate, sodium benzoate, sodium acetate, sodium chloride, starch, methyl cellulose, agar, bentonite, zanthan gum, and the like.

The dosage regimen for the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

In general, satisfactory results in animals are indicated to be obtained at a daily dosage of from about 0.1 to about 200 mg, preferably from about 0.1 to about 5 mg/kg animal body weight. In larger mammals, for example humans, an indicated daily dosage is in the range from about 0.5 to about 100 mg, preferably from about 1 to about 50 mg of an agent of the invention conveniently administered, for example, in divided doses up to four times a day or in sustained release form.

Injected intravenous, subcutaneous or intramuscular dosages of the compounds of the present invention, when used for the indicated effects, will range between about 0.001 to 1.0 mg/kg. Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Transdermal delivery can also be achieved using approaches known to those skilled in the art.

The subject invention further provides methods of synthesizing the unique and advantageous therapeutic compounds of the subject invention. Particularly, methods of producing less toxic therapeutic agents comprising introducing ester groups into therapeutic agents are taught. The ester linkage may be introduced into the compound at a site which is convenient in the manufacturing process for the compounds of the invention. Various exemplary synthetic routes for the preparation of the compounds of the subject invention are described. Additionally, the sensitivity of the ester linkage may be manipulated by the addition of side groups which hinder or promote the hydrolytic activity of the hydrolases or esterases responsible for cleaving the drug at the ester locus. Methods of adding such side groups, as well as the side groups themselves, are well known to the skilled artisan and can be readily carried out utilizing the guidance provided herein.

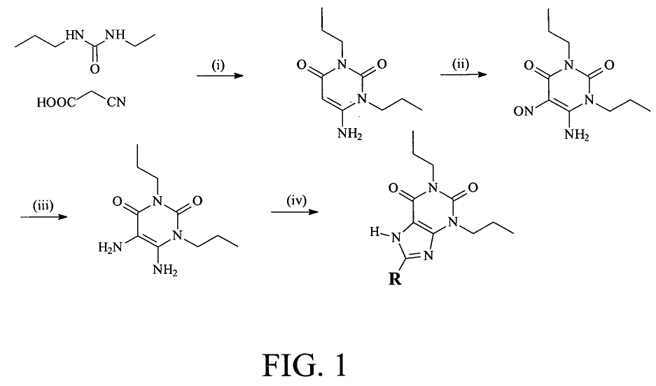

Examples of 1,3-dipropylxanthines of the subject invention having an ester function at the 8-position and a method of synthesizing them are described in FIG. 1.

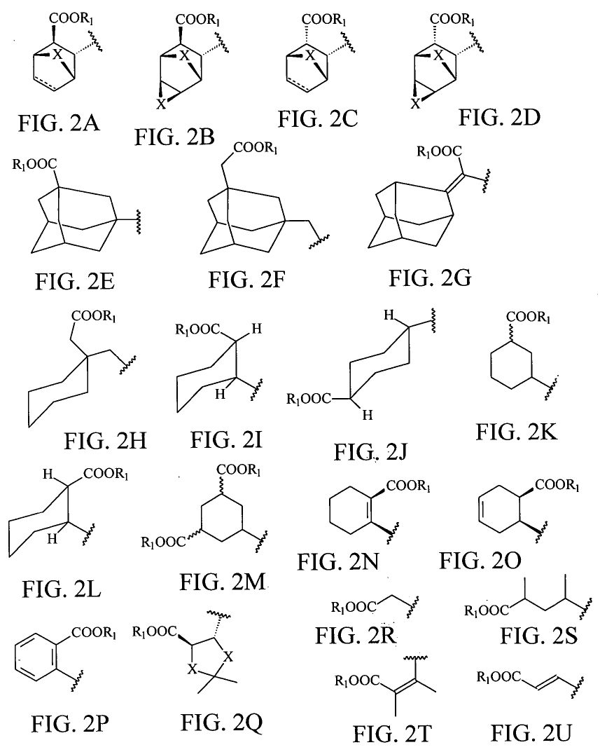

FIGS. 2A-2S show examples of compounds of the subject invention having AdoA₁R antagonist activity. R₁ is alkyl, including substituted (such as oxygenated) alkyl; X is CH₂ or O. Similar examples where R contains other cycloalkyl rings are also contemplated; for example cyclopentane, cyclopentene, cyclobutane, cyclobutene, cyclopropane.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. An A₁AdoR antagonist that has at least one characteristic chosen from the group consisting of:
a. the compound is metabolized both by CYP450 and by a non-oxidative metabolic enzyme or system of enzymes;
b. the compound has a short (up to four (4) hours) non-oxidative metabolic half-life;
c. the compound contains a hydrolysable bond that can be cleaved non-oxidatively by hydrolytic enzymes;
d. the primary metabolites of the compound result from the non-oxidative metabolism of the compound;
e. the primary metabolites are soluble in water at physiological pH;
f. the primary metabolites have negligible inhibitory activity at the IK_{R} (HERG) channel at normal therapeutic concentration of the parent drug in plasma;
g. the compound, as well as the metabolites thereof, does not cause metabolic DDI when co-administered with other drugs; and
h. the compound, as well as metabolites thereof, does not elevate LFT values when administered alone.

2. The compound, according to claim 1, which is a 1,3 dipropylxanthine with an ester function at the 8-position.

3. The compound, according to claim 1, wherein said ester function has a structure selected from the group consisting of: 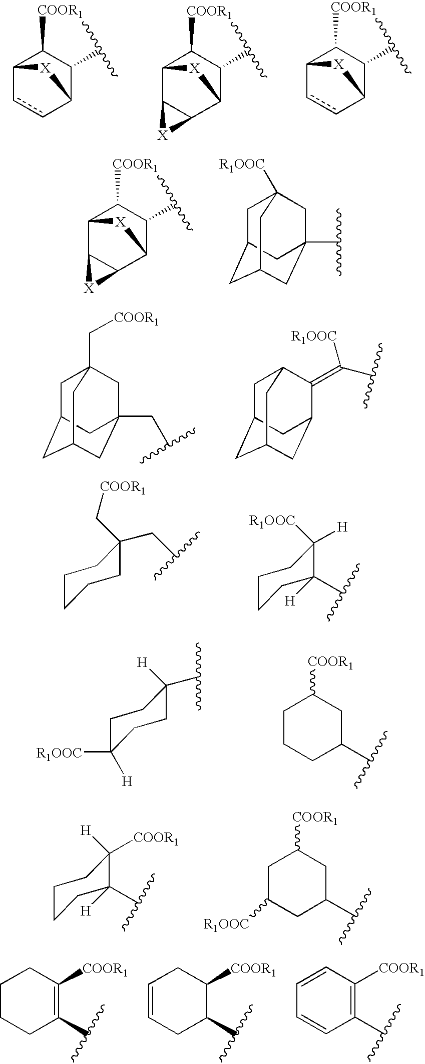 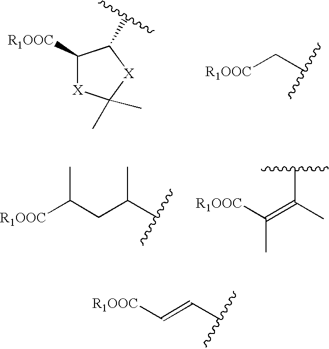
and salts thereof;
wherein R₁ is alkyl.

4. A pharmaceutical composition comprising an A₁AdoR antagonist that has at least one characteristic chosen from the group consisting of:
a. the compound is metabolized both by CYP450 and by a non-oxidative metabolic enzyme or system of enzymes;
b. the compound has a short (up to four (4) hours) non-oxidative metabolic half-life;
c. the compound contains a hydrolysable bond that can be cleaved non-oxidatively by hydrolytic enzymes;
d. the primary metabolites of the compound result from the non-oxidative metabolism of the compound;
e. the primary metabolites are soluble in water at physiological pH;
f. the primary metabolites have negligible inhibitory activity at the IK_{R} (HERG) channel at normal therapeutic concentration of the parent drug in plasma;
g. the compound, as well as the metabolites thereof, does not cause metabolic DDI when co-administered with other drugs; and
h. the compound, as well as metabolites thereof, does not elevate LFT values when administered alone;
wherein said composition further comprises a pharmaceutical carrier.

5. The pharmaceutical composition, according to claim 4, wherein said compound is a 1,3 dipropylxanthine that has an ester function at the 8-position.

6. The pharmaceutical composition, according to claim 4, wherein said ester function has a structure selected from the group consisting of: 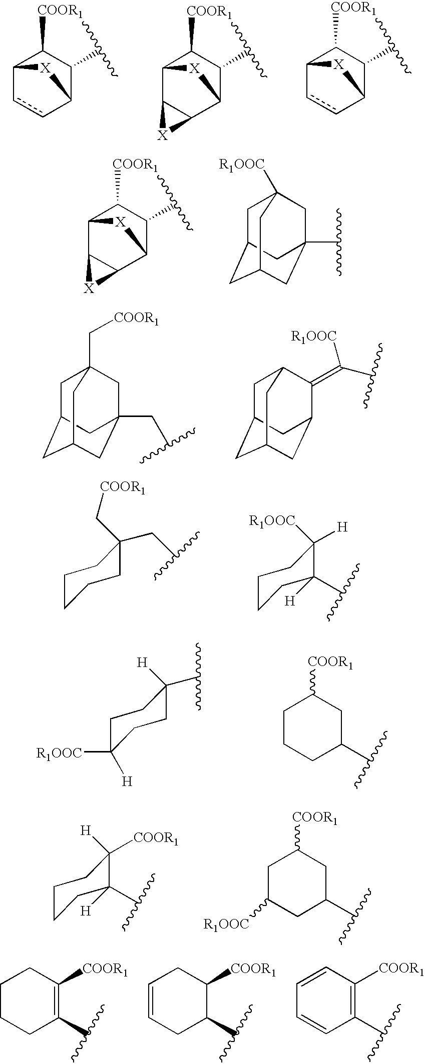 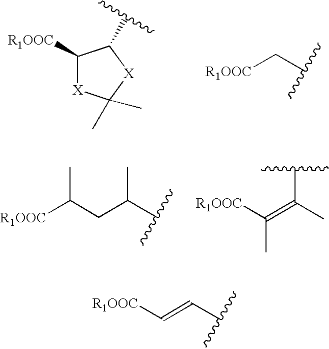
and salts thereof;
wherein R₁ is alkyl.

7. A method for inhibiting the A₁Ado receptor in an individual in need of such treatment wherein said method comprises administering to said individual a pharmaceutical composition comprising an A₁AdoR antagonist that has at least one characteristic chosen from the group consisting of:
a. the compound is metabolized both by CYP450 and by a non-oxidative metabolic enzyme or system of enzymes;
b. the compound has a short (up to four (4) hours) non-oxidative metabolic half-life;
c. the compound contains a hydrolysable bond that can be cleaved non-oxidatively by hydrolytic enzymes;
d. the primary metabolites of the compound result from the non-oxidative metabolism of the compound;
e. the primary metabolites are soluble in water at physiological pH;
f. the primary metabolites have negligible inhibitory activity at the IK_{R} (HERG) channel at normal therapeutic concentration of the parent drug in plasma;
g. the compound, as well as the metabolites thereof, does not cause metabolic DDI when co-administered with other drugs; and
h. the compound, as well as metabolites thereof, does not elevate LFT values when administered alone.

8. The method, according to claim 7, wherein said compound is a 1,3 dipropylxanthine with an ester function at the 8-position.

9. The method according to claim 7, wherein said ester function has a structure selected from the group consisting of: 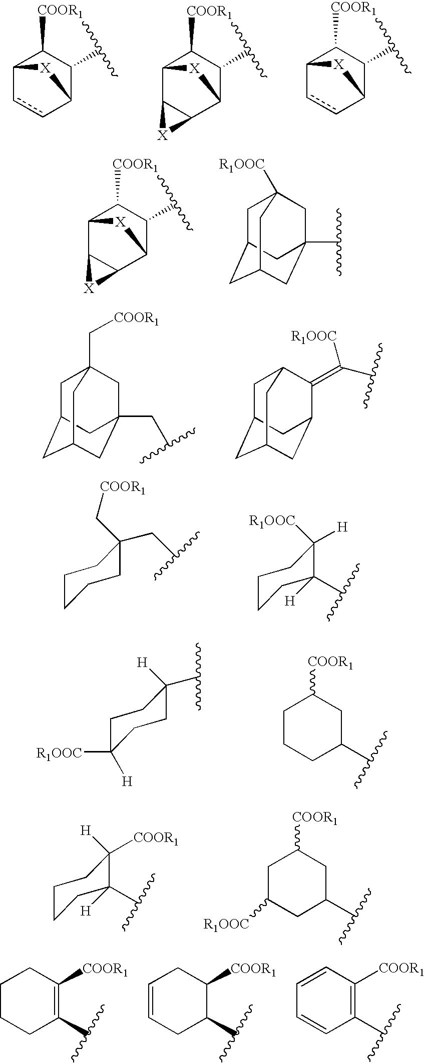 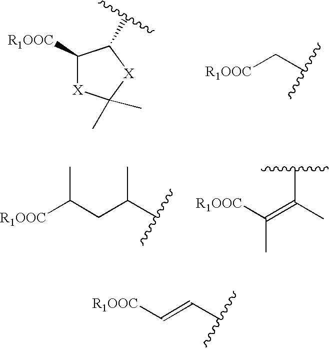
and salts thereof;
wherein R₁ is alkyl.

10. The method, according to claim 7, wherein the individual is a human.

11. The method, according to claim 7, wherein said individual has congestive heart failure.
